(19) United States
(12) Patent Application Publication
(10) Pub. No.: US 2002/0098584 A1
(43) Pub. Date: Jul. 25, 2002

(54) Postmortem stem cells

(76) Inventors: Theo D. Palmer, San Carlos, CA (US); Philip H. Schwartz, Irvine, CA (US); Philippe Taupin, La Jolla, CA (US); Fred H. Gage, La Jolla, CA (US)

Correspondence Address: KNOBBE MARTENS OLSON & BEAR LLP, 620 NEWPORT CENTER DRIVE, SIXTEENTH FLOOR, NEWPORT BEACH, CA, 92660, US

(21) Appl. No.: 10/012,885

(22) Filed: Nov. 6, 2001

Related U.S. Application Data
(60) Provisional application No. 60/246,314, filed on Nov. 6, 2000.

(51) Int. Cl. C12N 5/08

(52) U.S. Cl. 435/366; 435/384

(57) ABSTRACT

Disclosed are optimized methodologies for isolating and propagating stem cells from biopsies and postmortem tissues. Specifically disclosed are methods of culturing neural stem cells in the presence of a cocktail of trophic factors/co-factors for enhanced propagation.

### CROSS-REFERENCE TO RELATED APPLICATIONS

This application claims the benefit of U.S. Provisional Application No. 60/246,314, filed Nov. 6, 2000, which is incorporated herein by reference.

### STATEMENT OF RIGHTS TO INVENTIONS MADE UNDER FEDERALLY SPONSORED RESEARCH

Research relating to this invention was supported in part by contracts from the National Institute of Neurological Disorders and Stroke (NINDS) (NO1-NS-6-2348) and National Institute of Child Health and Human Development (NICHD) (NO1-HD-8-3284). The government may have certain rights in this invention.

### BACKGROUND OF THE INVENTION

1. Field of the Invention

This application relates to the in vitro growth of stem cells that has been isolated hours or days postmortem. In addition, multipotent stem cells can be isolated and exponentially expanded without losing the ability to correctly migrate and differentiate.

2. Description of the Related Art

The culture of neural precursors from the adult rodent brain has become routine (Alvarez-Buylla, et al. (1998) J. Neurobiol. 36, 105-110; Craig, et al. (1996) J. Neurosci. 16, 2649-2658; Palmer, et al. (1999) J. Neurosci. 19, 8487-8497; Reynolds, et al. (1992) Science 255, 1707-1710) and significant progress has been made in culturing neural precursors from human fetal tissues (Brannen, et al. (2000) Neuroreport 11, 1123-1128; Vescovi, A. L. et al. (1999) J. Neurotrauma 16, 689-693; Fricker, et al. (1999) J. Neurosci. 19, 5990-6005; Pincus, et al. (1998) Neurosurgery 42, 858-867; Moyer, et al. (1997) Transplant. Proc. 29, 2040-2041). Similar expansion of neural precursors from postnatal and adult human tissue has been problematic. Although precursors can be isolated and maintained in culture, yields are low and in vitro senescence limits the expansion of these cells once in culture (Pagano, et al. (2000) Stem Cells 18, 295-300).

There is exciting new evidence that hematopoietic progenitors may not be limited to the bone marrow microenvironment. Investigators at the University of Calgary have examined neuronal stem cells, which routinely differentiate along neuronal cell lineage pathways. When these cells were transplanted into lethally irradiated hosts, the investigators detected the presence of donor cell markers in newly produced myeloid and lymphoid cells (Bjornson (1999) Science 283:534). Investigators at the Baylor College of Medicine have performed similar studies using satellite cells isolated from murine skeletal muscle (Jackson et al. (1999) PNAS 96:14482). When these muscle-derived cells were transplanted into lethally irradiated hosts, the investigators detected the presence of the muscle gene markers in all blood cell lineages. Together, these studies indicate that neuronal and muscle tissues contain stem cells capable of hematopoietic differentiation. This suggest that sites other than the bone marrow may provide a renewable source of hematopoietic progenitors with potential application to human disease therapy (Quesenberry et al. (1999) J. Neurotrauma 16:661: Scheffler et al. (1999) Trends Neurosci 22:348; Svendson & Smith (1999) Trends Neurosci 22:357).

Just as neuronal and muscle cells are capable of regenerating the irradiated bone marrow, bone marrow derived cells are capable of repopulating other organ sites. When bone marrow derived hematopoietic and stromal cells are transplanted into an animal with an injured liver, they are capable of regenerating hepatic oval cells in the host animal (Peterse et al. (1999) Science 284:1168). Similarly, when labeled bone marrow stromal cells are implanted into the lateral ventricle of a neonatal mouse, they were capable of differentiating into mature astrocytes (Kopen et al. (1999) PNAS 96:10711). Indeed, when bone marrow stromal cells are transplanted intraperitoneally into mice, they are detected throughout the organs of the host animal, including the spleen, lung, bone marrow, bone, cartilage, and skin (Pereira et al (1998) PNAS 95:p 1142, 1998). These studies suggest that the bone marrow stromal cell is capable of differentiating into lineages different from their original dermal origin (Kopen et a. (1999) PNAS 96:10711).

CNS disorders encompass numerous afflictions such as neurodegenerative diseases (e.g. Alzheimer's and Parkinson's), acute brain injury (e.g. stroke, head injury, cerebral palsy) and a large number of CNS dysfunctions (e.g. depression, epilepsy, and schizophrenia). In recent years, neurodegenerative disease has become an important concern due to the expanding elderly population which is at greatest risk for these disorders. These diseases, which include Alzheimer's Disease, Multiple Sclerosis (MS), Huntington's Disease, Amyotrophic Lateral Sclerosis, and Parkinson's Disease, have been linked to the degeneration of neural cells in particular locations of the CNS, leading to the inability of these cells or the brain region to carry out their intended function.

In addition to neurodegenerative diseases, acute brain injuries often result in the loss of neural cells, the inappropriate functioning of the affected brain region, and subsequent behavior abnormalities. Probably the largest area of CNS dysfunction (with respect to the number of affected people) is not characterized by a loss of neural cells but rather by an abnormal functioning of existing neural cells. This may be due to inappropriate firing of neurons, or the abnormal synthesis, release, and processing of neurotransmitters. These dysfunctions may be the result of well studied and characterized disorders such as depression and epilepsy, or less understood disorders such as neurosis and psychosis.

Degeneration in a brain region known as the basal ganglia can lead to diseases with various cognitive and motor symptoms, depending on the exact location. The basal ganglia consists of many separate regions, including the striatum (which consists of the caudate and putamen), the globus pallidus, the substantia nigra, substantia innominate, ventral pallidum, nucleus basalis of Meynert, ventral tegmental area and the subthalamic nucleus.

In the case of Alzheimer's Disease, there is a profound cellular degeneration of the forebrain and cerebral cortex. In addition, upon closer inspection, a localized degeneration in an area of the basal ganglia, the nucleus basalis of Meynert, appears to be selectively degenerated. This nucleus normally sends cholinergic projections to the cerebral cortex which are thought to participate in cognitive functions including memory.

Many motor deficits are a result of degeneration in the basal ganglia. Huntington's Chorea is associated with the degeneration of neurons in the striatum, which leads to involuntary jerking movements in the host. Degeneration of a small region called the subthalamic nucleus is associated with violent flinging movements of the extremities in a condition called ballismus, while degeneration in the putamen and globus pallidus is associated with a condition of slow writhing movements or athetosis. In the case of Parkinson's Disease, degeneration is seen in another area of the basal ganglia, the substantia nigra par compacta. This area normally sends dopaminergic connections to the dorsal striatum which are important in regulating movement. Therapy for Parkinson's Disease has centered upon restoring dopaminergic activity to this circuit.

Other forms of neurological impairment can occur as a result of neural degeneration, such as amyotrophic lateral sclerosis and cerebral palsy, or as a result of CNS trauma, such as stroke and epilepsy.

Demyelination of central and peripheral neurons occurs in a number of pathologies and leads to improper signal conduction within the nervous systems. Myelin is a cellular sheath, formed by glial cells, that surrounds axons and axonal processes that enhances various electrochemical properties and provides trophic support to the neuron. Myelin is formed by Schwann cells in the PNS and by oligodendrocytes in the CNS. Among the various demyelinating diseases MS is the most notable.

To date, treatment for CNS disorders has been primarily via the administration of pharmaceutical compounds. Unfortunately, this type of treatment has been fraught with many complications including the limited ability to transport drugs across the blood-brain barrier and the drug-tolerance which is acquired by patients to whom these drugs are administered long-term. For instance, partial restoration of dopaminergic activity in Parkinson's patients has been achieved with levodopa, which is a dopamine precursor able to cross the blood-brain barrier. However, patients become tolerant to the effects of levodopa, and therefore, steadily increasing dosages are needed to maintain its effects. In addition, there are a number of side effects associated with levodopa such as increased and uncontrollable movement.

The infection of neurons with foreign genes and implantation into the CNS would be ideal due to their ability to extend processes, make synapses and be regulated by the environment. However, differentiated neurons do not divide and transfection with foreign genes by chemical and physical means is not efficient, nor are they stable for long periods of time. The infection of primary neuronal precursors with retroviral vectors in vitro is not practical either because neuroblasts are intrinsically controlled to undergo a limited number of divisions making the selection of a large number of neurons, that incorporate and express the foreign gene, nearly impossible. The possibility of immortalizing the neuronal precursors by retroviral transfer of oncogenes and their subsequent infection of a desired gene is not preferred due to the potential for tumor formation by the implanted cells.

Recently, the concept of neurological tissue grafting has been applied to the treatment of neurological diseases such as Parkinson's Disease. Neural grafts may avert the need not only for constant drug administration, but also for complicated drug delivery systems which arise due to the blood-brain barrier. However, there are limitations to this technique as well. First, cells used for transplantation which carry cell surface molecules of a differentiated cell from another host can induce an immune reaction in the host. In addition, the cells must be at a stage of development where they are able to form normal neural connections with neighboring cells. For these reasons, initial studies on neurotransplantation centered on the use of fetal cells. Perlow, et al. describe the transplantation of fetal dopaminergic neurons into adult rats with chemically induced nigrostriatal lesions in “Brain grafts reduce motor abnormalities produced by destruction of nigrostriatal dopamine system,” Science 204:643-647 (1979). These grafts showed good survival, axonal outgrowth and significantly reduced the motor abnormalities in the host animals.

It would be more preferable to have a well-defined, reproducible source of neural tissue for transplantation that is available in unlimited amounts. Since adult neural tissue undergoes minimal division, it does not readily meet these criteria. While astrocytes retain the ability to divide and are probably amenable to infection with foreign genes, their ability to form synapses with neuronal cells is limited and consequently so is their extrinsic regulation of the expression and release of the foreign gene product.

Oligodendrocytes suffer from some of the same problems. In addition, mature oligodendrocytes do not divide, limiting the infection of oligodendrocytes to their progenitor cells (e.g. 0-2A cells). However, due to the limited proliferative ability of oligodendrocyte progenitors, the infection and harvesting of these cells does not represent a practical source.

In addition to the need for a well-defined, reproducible source of neural cells available in unlimited amounts for transplantation purposes, a similar need exists for drug screening purposes and for the study of CNS function, dysfunction, and development. The mature human nervous system is composed of billions of cells that are generated during development from a small number of precursors located in the neural tube. Due to the complexity of the mammalian CNS, the study of CNS developmental pathways, as well as alterations that occur in adult mammalian CNS due to dysfunction, has been difficult. Such areas would be better studied using relatively simple models of the CNS under defined conditions.

Here we describe an optimized methodology for isolating and propagating precursors from biopsies and postmortem tissues. These methods improve cell yield and facilitate expansion of immature cells capable of generating neurons and glia in vitro.

### SUMMARY OF THE INVENTION

Multipotent stem cells cells can be isolated from postmortem tissue and exponentially expanded without losing the ability to correctly migrate and differentiate. Recent work using stem cells from postmortem neural tissue shows that human stem cells (including progenitor cells) can be propagated and transplanted. In the present work, we describe methodological advances that allow the routine purification of neural stem cells from biopsy material and postmortem tissues.

Under ideal conditions, precursors from postnatal and adult brain can be exponentially expanded as monolayer cultures, cryopreserved and recultured for up to about 40 population doublings prior to reaching senescence. In the most dramatic instances, proliferative stem cells were efficiently isolated and expanded from numerous brain regions at more than 20 hours postmortem. In addition, similar cultures can be initiated from cryopreserved postmortem tissues with only moderate losses in cell recovery.

The fact that human precursors were still viable at such late postmortem intervals suggests that stem cells, especially neural stem cells/precursors, are uniquely resistant to postmortemischemic and oxidative stress. These observations provide an important extension to the tissue resources available for numerous uses, including for drug screening, diagnostics, genomics, transplantation, as well as to study the natural behavior and repair potential of stem cells present in the developing, postnatal, and adult human brain. Accordingly, herein are provided novel methods for the isolation and propagation of stem cells, and kits comprising the stem cells and/or the media supporting the propagation thereof.

### BRIEF DESCRIPTION OF THE DRAWINGS

FIG. 1A-C are bar graphs showing the percent of brain cells in a population that are immunopositive for markers for Neurons (Tuj-1, NeuN), Astrocytes (glial fibrillary acidic protein (GFAP)), and Oligodendrocytes (O4). All three cell types are detectable in cultures from fetal (1A), newborn (1B) or adult (1C) brain tissues.

FIGS. 2A-C are line graphs showing the number of cells and cell doublings in primary cell cultures from fetal (2A), newborn (2B) or adult (2C) brain tissues, and reveal stable growth rates up to the point of senescence.

### DETAILED DESCRIPTION OF THE PREFERRED EMBODIMENT

Disclosed herein are methods to isolate and propogate stem cells from postmortem human subjects. The invention describes the isolation, proliferation and reintroduction of stem cells from a variety of tissue (see, e.g., Palmer, Nature 2001 (411) 42-43) incorporated by reference herein).

A “stem cell” (which is interchanged with the term “progenitor cells”) as used herein is a undifferentiated cell which is capable of essentially unlimited propagation either in vivo or ex vivo and capable of differentiation to other cell types. This can include certain differentiated, committed, immature, progenitor, precursor, or mature cell types present in the tissue from which it was isolated, or dramatically differentiated cell types, such as for example the erythrocytes and lymphocytes that derive from a common precursor cell, or even to cell types at any stage in a tissue completely different from the tissue from which the stem cell is obtained. Certain stem cells are pluripotential, and given the appropriate signals from their environment, they can differentiate into any tissue in the body. For example, blood stem cells may become brain cells or liver cells, neural stem cells can become blood cells, such that. In general, stem cells refer to cells that are self-renewing and multipotent (i.e., that are not lineage restricted) and able to differentiate, whereas “progenitor” or “precursor” cells refer to undifferentiated cells whose lineal descendants differentiate along the appropriate pathway to produce a fully differentiated phenotype (i.e., cells with a restricted lineage). For example, neural stem cells isolated from the hippocampus (HC) or the subventricular zone, are self renewing and able to generate, in vitro, multiple types of cells including neurons, glia and even hematopoetic cells.

Accordingly, provided herein are methods for propagating stem cells (including progenitor and precursor cells) in a cocktail of trophic factors and/or co-trophic factors, and kits comprising the cocktail media, alone or in combination with the stem cells.

It should be realized that although the methods in the Examples described below relate to isolation of stem cells from postmortem human central nervous system, it is anticipated that these same techniques can be used to isolate and propagate stem cells from any tissue, including the brain, heart, liver, lung, bone marrow, and the like. Indeed, it is expected that any tissue can yield progenitor and stem cells if processed in the manner described herein. In a presently preferred embodiment, the stem cells are isolated from the postmortem CNS of a postnatal human subject, more preferably from the adult postmortem CNS. CNS tissue from which stem cells can be derived include whole brain, hippocampus, spinal cord, cortex, striatum, cerebellum, thalamus, hypothalamus, amigdyla, basal forebrain, ventral mesencephalon, optic nerve, locus ceruleus, and the like.

As used herein, the postmortem neural stem cells can be cultivated in the presence of a trophic factor/co-factors, or combinations of trophic factors/co-factors. As used herein, the term “trophic factor” refers to compounds with trophic actions that promote and/or control proliferation, differentiation, migration, survival and/or death (e.g., apoptosis) of their target cells. Such factors include cytokines, neurotrophins, growth factors, mitogens, co-factors, and the like, including epidermal growth factor (EGF), fibroblast growth factor (FGF), platelet-derived growth factor (PDGF), insulin-like growth factors, ciliary neurotrophic factor and related molecules, glial-derived growth factor and related molecules, schwanoma-derived growth factor, glial growth factor, stiatal-derived neuronotrophic factor, hepatocyte growth factor, scatter factor (HGF-SF), transforming growth factor-beta and related molecules, neurotransmitters, and hormones.

“Trophic factors” have a broad range of biological activities and their activity and specificity may be achieved by cooperation with other factors, including co-factors therefore. Although trophic factors are generally active at extremely low concentrations, high concentrations of mitogen together with high cell density are often preferred to induce proliferation of multipotent neural stem cell populations.

Preferred trophic factors contemplated for use in stimulating stem cells are mitogenic growth factors, like FGF-2 (Gage, F. H., et al., 1995, Proc. Natl Acad. Sci. USA 92:11879-11883) and epidermal growth factor (EGF) (Lois, C., and Alvarez-Buylla, A., 1993, Proc. Natl. Acad. Sci. USA 90(5):2074-2077), which induce proliferation and/or propagation of stem cells, e.g., neural stem cells isolated from the brain. Studies from single cells in culture demonstrate that FGF-2 (Gritti, A., et al., 1996, J. Neurosci. 16:1091-1100) and EGF (Reynolds, B. A., and Weiss, S., 1996, Develop. Biol. 175:1-13) are mitogens for multipotent neural stem cells and likely cooperate with other trophic factors (Cattaneo, E., and McKay, R., 1990, Nature 347:762-765; Stemple, D. L., and Anderson, D. J., 1992, Cell 71:973-985), some of which are yet unknown (Davis, A. A., and Temple, S., 1994, Nature 372:263-266; Temple, S., 1989, Nature 340:471-473; Kilpatrick, T. J., and Bartlett, P. F., 1993, Neuron 10:255-265; Palmer, T. D., et al., 1997, Mol. Cell. Neurosci. 8:389-404) to achieve specificity.

Those of ordinary skill in the art will recognize additional trophic factors that can be used to stimulate stem cells (see, e.g., Aebischer et al. Neurotrophic Factors (Handbook of Experimental Pharmacology, Vol 134) (Springer Verlag, 1998); Meyers, R. A. Encyclopedia of Molecular Biology and Molecular Medicine: Denaturation of DNA Growth Factors (VCH Pub, 1996); Meager & Robinson, Growth Factors: Essential Data (John Wiley and Sons, 1999); McKay & Brown, Growth Factors and Receptors: A Practical Approach (Oxford University Press, 1998); Leroith & Bondy, Growth Factors and Cytokines in Health and Disease, Vol 1A and 1B : A Multi-Volume Treatise (JAI Pr, 1996); Lenfant et al., Growth Factors of the Vascular and Nervous Systems: Functional Characterization and Biotechnology: International Symposium on Biotechnology of Grow (S. Karger Publishing, 1992).

For example, following isolation of the postmortem tissue, these cells can be cultivated in medium having “neurotrophins” (or “neurotrophic factor”) that promote the survival and functional activity of nerve or glial cells, including a factor that enhances neural differentiation, induces neural proliferation, influences synaptic functions, and/or promotes the survival of neurons that are normally destined to die, during different phases of the development of the central and peripheral nervous system.

Exemplary neurotrophins include, for example, ciliary neurotrophic factor (CNF), nerve growth factor (NGF), FGF, brain-derived neurotrophic factor (BDNF), Neurotrophin-3 (NT-3), glia derived neurotrophic factor (GDNF), and the like. Such factors are characterized by their trophic actions, their expression patterns in the brain, and molecular aspects of their receptors and intracellular signaling pathways. Neurotrophic factors that have been identified include NT-4, NT-5, NT-6, NT-7, ciliary neuronotrophic factor (CNTF), GDNF, and Purpurin. Neuron-specific enolase (NSE) has been found to be a neuronal survival factor. Other factors possessing a broader spectrum of functions, which have neurotrophic activities but are not normally classified as neurotrophins, also are contemplated for use in the invention.

These “neurotrophin-like factors” include epidermal growth factor (EGF), heparin-binding neurite-promoting factor (HBNF), insulin-like growth factor 2 (IGF-2), aFGF and b-FGF , platelet derived growth factor (PDGF), NSE, and Activin A. Other factors have been identified which specifically influence neuronal differentiation and influence transmitter phenotypes without affecting neuronal survival. Although the intracerebral administration of FGF-2 has been shown to stimulate neurogenesis in the adult rat subventricular zone, FGF-2 alone in the adult rat hippocampus has a limited effect on the proliferation of neural stem/progenitor cells (Kuhn et al. (1997); Wagner et al. (1999) each herein incorporated by reference).

In a preferred embodiment, neural stem cells can be cultured in FGF and FGF-like factors, including a-FGF, b-FGF such as FGF-2, FGF-4, FGF-6, and the like. A particularly advantageous medium for culturing neural stem cells comprises the following: FGF alone (particularly basic FGF or FGF-2), EGF and/or PDGF, and at least a cofactor for at least one of the neurotrophins.

As used herein, “co-factors” refers to molecules which stimulate and/or potentiate the trophic factor activity and/or specificity. This was clearly identified in low density cells where trophic factors are unable, or at best, at minimal levels, able to proliferate undifferentiated cells without a co-factor. One particular such co-factor is the composition, glycosylated cystatin C (CCg), an neurotrophin co-factor, such as FGF, that stimulates proliferation of neural and fibroblast associated undifferentiated cells. CCg has been identified to co-stimulate FGF, as well as trophic factors independent of FGF. CCg acts in cooperation with basic fibroblast growth factor (FGF-2) to induce neural stem/progenitor cell proliferation. (See, for example, Gage et al., WO00/33791).

In a preferred embodiment, the stem cells are propagated in culture comprising a cocktail of trophic factors/co-factors, in a base media, in vitro. Non-limiting examples of base media useful in the methods of the invention include Minimum Essential Medium Eagle, ADC-1, LPM (Bovine Serum Albumin-free), F10(HAM), F12 (HAM), DCCM1, DCCM2, RPMI 1640, BGJ. Medium (with and without Fitton-Jackson Modification), Basal Medium Eagle (BME-with the addition of Earle's salt base), Dulbecco's Modified Eagle Medium (DMEM-without serum), Yamane, IMEM-20, Glasgow Modification Eagle Medium (GMEM), Leibovitz L-15 Medium, McCoy's 5A Medium, Medium M199 (M199E-with Earle's sale base), Medium M199 (M199H-with Hank's salt base), Minimum Essential Medium Eagle (MEM-E-with Earle's salt base), Minimum Essential Medium Eagle (MEM-H-with Hank's salt base) and Minimum Essential Medium Eagle (MEM-NAA with non essential amino acids), among numerous others, including medium 199, CMRL 1415, CMRL 1969, CMRL 1066, NCTC 135, MB 75261, MAB 8713, DM 145, Williams' G, Neuman & Tytell, Higuchi, MCDB 301, MCDB 202, MCDB 501, MCDB 401, MCDB 411, MDBC 153. A preferred medium for use in the present invention is DMEM. These and other useful media are available from GIBCO, Grand Island, N.Y., USA and Biological Industries, Bet HaEmek, Israel, among others. A number of these media are summarized in Methods in Enzymology, Volume LVIII, “Cell Culture”, pp. 62-72, edited by William B. Jakoby and Ira H. Pastan, published by Academic Press, Inc.

Additional non-limiting examples of media useful in the methods of the invention can contain fetal serum of bovine (e.g., BIT-9500 (bovine serum albumin, transferring, insulin: Stem Cell technologies)) or other species at a concentration of at least 1% to about 30%, preferably at least about 5% to 15%, mostly preferably about 10%. Embryonic extract of chicken or other species can be present at a concentration of about 1% to 30%, preferably at least about 5% to 15%, most preferably about 10%. Those of skill in the art will readily recognize media suitable for propagation of human stem cells. For example, progesterone is an undesirable component in such media, whereas certain other growth factors, cytokines and hormones may be desirable.

By “growth factors, cytokines, hormones” is intended the following specific factors including, but not limited to, growth hormone, erythropoeitin, thrombopoietin, interleukin 3, interleukin 6, interleukin 7, macrophage colony stimulating factor, c-kit ligand/stem cell factor, osteoprotegerin ligand, insulin, insulin like growth factors, epidermal growth factor, fibroblast growth factor, nerve growth factor, cilary neurotrophic factor, platelet derived growth factor, and bone morphogenetic protein at concentrations of between pigogram/ml to milligram/ml levels. At such concentrations, the growth factors, cytokines and hormones useful in the methods of the invention are able to induce, up to 100% the formation of blood cells (lymphoid, erythroid, myeloid or platelet lineages) from adipose derived stromal cells in colony forming unit (CFU) assays. (Moore et al. (1973) J. Natl. Cancer Inst. 50:603-623; Lee et al. (1989) J. Immunol. 142:3875-3883; Medina et al. (2993) J. Exp. Med. 178:1507-1515. Hormones that provide spatial cues include thyroid hormone and the like. Receptors include the steroid/thyroid hormone superfamily of receptors, neurotrophin receptors TrkB and TrkC, and the like. Other components will be readily recognized, e.g., transferring, insulin, and the like.

The term “Isolating” a stem cell refers to the process of removing a stem cell from a tissue sample and separating away other cells which are not stem cells of the tissue. An isolated stem cell will be generally free from contamination by other cell types and will generally have the capability of propagation and differentiation to produce mature cells of the tissue from which it was isolated. However, when dealing with a collection of stem cells, e.g., a culture of stem cells, it is understood that it may be practically impossible to obtain a collection of stem cells which is 100% pure. Therefore, an isolated stem cell can exist in the presence of a small fraction of other cell types which do not interfere with the utilization of the stem cell for analysis or production of other, differentiated cell types. Isolated stem cells will generally be at least 30%, 40%, 50%, 60%, 70%, 80%, 85%, 90%, 95%, 98%, or 99% pure. Preferably, isolated stem cells according to the invention will be at least 98% or at least 99% pure. See for example, Smith, et al., U.S. Pat. No. 6,146,888.

As will be appreciated by those of skill in the art, proper isolation and treatment of source tissues for invention stem cells is desirable in order to obtain a population of cells comprising invention stem cells. Thus, while a whole brain or other source neuronal tissue, as described herein, all comprise stem cells, it is desirable for therapeutic purposes to provide a cell population containing primarily isolated stem cells and lacking a substantial amount of other cell types and/or debris. This enrichment can be carried out by a number of methods. Thus, provided herein are methods for enriching a cell population containing stem cells for such stem cells, said method comprising subjecting dissociated mammalian CNS tissue to one or more separation systems. Although it is contemplated that, with proper execution, any separation system can be adapted to isolate stem cells from such tissue, examples of useful separation systems include physical separation, magnetic separation, using antibody-coated magnetic beads, affinity chromatography, cytotoxic agents joined to a monoclonal antibody or used in conjunction with a monoclonal antibody, including, but not limited to, complement and cytotoxins, and “panning” with antibody attached to a solid matrix, e.g., plate, elutriation or any other convenient technique, buoyancy-based separation systems, charge-based separation systems, fluorescent activated cell sorting systems (FACS), and the like, as well as combinations thereof.

A stem cell is “propagated” when it is expanded in culture and gives rise by cell division to other stem cells and/or progenitor cells. Expansion of stem cells, as described herein, occur as stem cells proliferate in a culture in the presence of certain growth conditions as described herein. “Essentially unlimited propagation” can be determined, for example, by the ability of an isolated stem cell to be propagated through at least 50, preferably 100, and even up to 200 or more cell divisions in a cell culture system. Those of skill in the art also refer to certain stem cells as “totipotent,” meaning that they can give rise to all the cells of an organism as for germ cells, or or “pluripotent,” meaning that they can give rise to many different cell types, but not all the cells of an organism. When a stem cell differentiates it generally gives rise to a more adult cell type, which may be a partially differentiated cell such as a progenitor cell, a differentiated cell, or a terminally differentiated cell. Stem cells of the present invention can be highly motile.

The temporal and spatial cues described herein to differentiate the cells may be provided to cells as either molecules that are supplied exogenously (i.e., extracellularly) or endogenously (e.g., through the expression of native and/or introduced nucleic acids encoding such molecules, and the like). Those of skill in the art will readily recognize the cues necessary to differentiate the stem cells to the desired fate. As used herein with respect to stem cells, “heterotypic environments” to which the cells are able to adapt include all non-source, or non-native, tissue. For example, neural and blood stem cells can adapt to differentiate into whole brain, hippocampus, spinal cord, cortex, striatum, cerebellum, thalamus, hypothalamus, amigdyla, basal forebrain, ventral mesencephalon, optic nerve, locus ceruleus, and the like, as well as CNS associated tissues such as eye tissues, the vitreous of the eye, and the like. In addition, heterotypic environments include in vitro culture systems in which the foregoing cell types and lineages derived therefrom are cultured. The presence of the differentiated cells may be detected in a subject by a variety of techniques including, but not limited to, flow cytometry, immunohistochemical, in situ hybridization, and/or other histologic or cellular biologic techniques. See, for example, Kopen et al., (1999) Proc Natl Acad Sci 96:10711-10716.

The human stem cells of this invention have numerous uses, including for drug screening, diagnostics, genomics and transplantation.

The invention stem cells, once proliferated in vitro or in culture, are self-renewing (i.e., are capable of replication to generate additional stem cells). In addition, the invention stem cells, due to their pluripotent character, are capable of exhibiting a wide variety of responses characteristic of stem cells. Because the invention stem cells are pluripotent, invention stem cells response to a heterotypic environment including differentiation into a more lineage restricted type of cell found in the tissue from which the invention stem cells was isolated. For example, neural stem cells response to a neural heterotypic environment includes differentiation into neurons, and glia, including astroglia and/or oligodendroglia, and the like.

As a result of the remarkable ability of the invention stem cells to adapt to a variety of heterotypic environments with the concomitant ability to integrate and differentiate, they are excellent candidates for gene therapy applications. Accordingly, provided herein are invention stem cells containing one or more heterologous DNA sequences (e.g., transgenes, and the like). In a presently preferred embodiment, the invention stem cells are capable of expressing proteins encoded by the heterologous DNA sequences.

As described herein, invention stem cells are able to integrate and differentiate into a number of different tissue types. Preferably, neural stem cells will be employed to integrate and differentiate primarily into neural tissues. As such, the invention stem cells are useful as therapeutic agents for replacing or augmenting diseased or damaged tissue. In addition the invention stem cells may, however, also carry and express heterologous DNA sequences.

The cells and methods of this invention are intended for use in a mammalian host, recipient, patient, subject or individual, preferably a primate, most preferably a human.

The cells and methods of this invention may be useful in the treatment of various neurodegenerative diseases and other disorders. It is contemplated that the cells will replace diseased, damaged or lost tissue in the host. Alternatively, the transplanted tissue may augment the function of the endogenous affected host tissue. The transplanted cells may also be genetically engineered to provide a biologically active molecule that is therapeutically effective.

Thus, provided herein are methods of therapy comprising administering to a patient in need thereof a cell population comprising modified stem cells, such as, for example, those described herein, in an amount sufficient to provide a desired therapeutic effect. As those of skill in the art will understand, an amount sufficient to provide a therapeutic effect will vary according to the condition being treated, the locus of introduction, the level of enrichment for stem cells in the donor cell population, the presence in donor stem cells of transgenes, the relative level of expression of any such transgene(s), and the like.

Accordingly, the individual practitioner may be required to take such factors into account when proceeding with a therapeutic regimen comprising of administering to a patient in need a cell population consisting of the invention stem cells. A “therapeutically effective amount” is an amount effective for introducing or complementing one or more missing and/or defective genes, wherein the gene(s) so introduced comprise heterologous genetic material contained and expressed within said stem cells and their descendants.

The cells may be administered into a host in order in a wide variety of ways. Preferred modes of administration are parenteral, intraperitoneal, intravenous, intradermal, epidural, intraspinal, intrastemal, intra-articular, intra-synovial, intrathecal, intra-arterial, intracardiac, intramuscular, intranasal, subcutaneous, intraorbital, intracapsular, topical, transdermal patch, via rectal, vaginal or urethral administration including via suppository, percutaneous, nasal spray, surgical implant, internal surgical paint, infusion pump, or via catheter. In one embodiment, the agent and carrier are administered in a slow release formulation such as a direct tissue injection or bolus, implant, microparticle, microsphere, nanoparticle or nanosphere.

The cells of this invention may be transplanted “naked” into patients according to conventional techniques, into the CNS, as described for example, in U.S. Pat. Nos. 5,082,670 and 5,618,531, each incorporated herein by reference, or into any other suitable site in the body.

In one embodiment, the human stem cells are transplanted directly into the CNS. Parenchymal and intrathecal sites are contemplated. It will be appreciated that the exact location in the CNS will vary according to the disease state. According to one aspect of this invention, provided herein is methodology for improving the viability of transplanted human neural stem cells. The cells may also be encapsulated and used to deliver biologically active molecules, according to known encapsulation technologies, including microencapsulation (see, e.g., U.S. Pat. Nos. 4,352,883; 4,353,888; and 5,084,350, herein incorporated by reference), (b) macroencapsulation (see, e.g., U.S. Pat. Nos. 5,284,761, 5,158,881, 4,976,859 and 4,968,733 and published PCT patent applications WO92/19195, WO 95/05452, each incorporated herein by reference), and macroencapsulation, as described in U.S. Pat. Nos. 5,284,761, 5,158,881, 4,976,859 and 4,968,733 and published PCT patent applications WO92/19195, WO 95/05452, each incorporated herein by reference.

These cells find use in regenerating the hematopoietic system of a host deficient in any class of hematopoietic cells; a host that is diseased and can be treated by removal of blood marrow, isolation of stem cells, and treatment with drugs or irradiation prior to re-engraftment of stem cells; producing various hematopoietic cells; detecting and evaluating growth factors relevant to stem cell self-regeneration; and the development of hematopoietic cell lineages and assaying for factors associated with hematopoietic development.

The hematopoietic cells of the invention find use in therapy for a variety of disorders. Particularly, disorders associated with blood, marrow, stem cells, etc. are of interest. The transformed cells may be used to treat or prevent HIV infection.

Disorders that can be treated by infusion of the disclosed cells include, but are not limited to, diseases resulting from a failure of a dysfunction of normal blood cell production and maturation (i.e., aplastic anemia and hypoproliferative stem cell disorders); neoplastic, malignant diseases in the hematopoietic organs (e.g., leukemia and lymphomas); broad spectrum malignant solid tumors of non-hematopoietic origin; autoimmune conditions; and genetic disorders. Such disorders include, but are not limited to diseases resulting from a failure or dysfunction of normal blood cell production and maturation hyperproliferative stem cell disorders, including aplastic anemia, pancytopenia, agranulocytosis, thrombocytopenia, red cell aplasia, Blackfan-Diamond syndrome, due to drugs, radiation, or infection, idiopathic; hematopoietic malignancies including acute lymphoblastic (lymphocytic) leukemia, chronic lymphocytic leukemia, acute myelogenous leukemia, chronic myelogenous leukemia, acute malignant myelosclerosis, multiple myeloma, polycythemia vera, agnogenic myelometaplasia, Waldenstrom's macroglobulinemia, Hodgkin's lymphoma, non-Hodgkin's lymphoma; immunosuppression in patients with malignant, solid tumors including malignant melanoma, carcinoma of the stomach, ovarian carcinoma, breast carcinoma, small cell lung carcinoma, retinoblastoma, testicular carcinoma, glioblastoma, rhabdomyosarcoma, neuroblastoma, Ewing's sarcoma, lymphoma; autoimmune diseases including rheumatoid arthritis, diabetes type I, chronic hepatitis, multiple sclerosis, systemic lupus erythematosus; genetic (congenital) disorders including anemias, familial aplastic, Fanconi's syndrome, dihydrofolate reductase deficiencies, formamino transferase deficiency, Lesch-Nyhan syndrome, congenital dyserythropoietic syndrome I-IV, Chwachmann-Diamond syndrome, dihydrofolate reductase deficiencies, formamino transferase deficiency, Lesch-Nyhan syndrome, congenital spherocytosis, congenital elliptocytosis, congenital stomatocytosis, congenital Rh null disease, paroxysmal nocturnal hemoglobinuria, G6PD (glucose-6-phhosphate dehydrogenase) variants 1, 2, 3, pyruvate kinase deficiency, congenital erythropoietin sensitivity, deficiency, sickle cell disease and trait, thalassemia alpha, beta, gamma, met-hemoglobinemia, congenital disorders of immunity, severe combined immunodeficiency disease (SCID), bare lymphocyte syndrome, ionophore-responsive combined immunodeficiency, combined immunodeficiency with a capping abnormality, nucleoside phosphorylase deficiency, granulocyte actin deficiency, infantile agranulocytosis, Gaucher's disease, adenosine deaminase deficiency, Kostmann's syndrome, reticular dysgenesis, congenital Leukocyte dysfunction syndromes; and others such as osteoporosis, myelosclerosis, acquired hemolytic anemias, acquired immunodeficiencies, infectious disorders causing primary or secondary immunodeficiencies, bacterial infections (e.g., Brucellosis, Listerosis, tuberculosis, leprosy), parasitic infections (e.g., malaria, Leishmaniasis), fungal infections, disorders involving disproportionsin lymphoid cell sets and impaired immune functions due to aging, phagocyte disorders, Kostmann's agranulocytosis, chronic granulomatous disease, Chediak-Higachi syndrome, neutrophil actin deficiency, neutrophil membrane GP-180 deficiency, metabolic storage diseases, mucopolysaccharidoses, mucolipidoses, miscellaneous disorders involving immune mechanisms, Wiskott-Aldrich Syndrome, alpha 1-antirypsin deficiency, etc.

Diseases or pathologies include neurodegenerative diseases, hepatodegenerative diseases, nephrodegenerative disease, spinal cord injury, head trauma or surgery, viral infections that result in tissue, organ, or gland degeneration, and the like. Such neurodegenerative diseases include but are not limited to, AIDS dementia complex; demyeliriating diseases, such as multiple sclerosis and acute transferase myelitis; extrapyramidal and cerebellar disorders, such as lesions of the ecorticospinal system; disorders of the basal ganglia or cerebellar disorders; hyperkinetic movement disorders, such as Huntington's Chorea and senile chorea; drug- induced movement disorders, such as those induced by drugs that block CNS dopamine receptors; hypokinetic movement disorders, such as Parkinson's disease; progressive supra-nucleo palsy; structural lesions of the cerebellum; spinocerebellar degenerations, such as spinal ataxia, Friedreich's ataxia, cerebellar cortical degenerations, multiple systems degenerations (Mencel, Dejerine Thomas, Shi-Drager, and Machado-Joseph), systermioc disorders, such as Rufsum's disease, abetalipoprotemia, ataxia, telangiectasia; and mitochondrial multi-system disorder; demyelinating core disorders, such as multiple sclerosis, acute transverse myelitis; and disorders of the motor unit, such as neurogenic muscular atrophies (anterior horn cell degeneration, such as amyotrophic lateral sclerosis, infantile spinal muscular atrophy and juvenile spinal muscular atrophy); Alzheimer's disease; Down's Syndrome in middle age; Diffuse Lewy body disease; Senile Demetia of Lewy body type; Wernicke-Korsakoff syndrome; chronic alcoholism; Creutzfeldt-Jakob disease; Subacute sclerosing panencephalitis hallerrorden-Spatz disease; and Dementia pugilistica. See, e.g., Berkow et. al., (eds.) (1987), The Merck Manual, (15.sup.th) ed.), Merck and Co., Rahway, N.J., which reference, and references cited therein, are entirely incorporated herein by reference.

The cells of this invention are also contemplated in the treatment of various demyelinating and dysmyelinating disorders, such as Pelizaeus-Merzbacher disease, multiple sclerosis, various leukodystrophies, as well as various neuritis and neuropathies, particularly of the eye. We contemplate using cell cultures enriched in oligodendrocytes or oligodendrocyte precursor or progenitors, such cultures prepared and transplanted according to this invention to promote remyelination of demyelinated areas in the host.

The cells of this invention are also contemplated in the treatment of various acute and chronic pains, as well as for certain nerve regeneration applications (such as spinal cord injury). We also contemplate use of human stem cells for use in sparing or sprouting of photoreceptors in the eye.

Although the retina originates from the neural tube, the optic vesicle forms early in development and the retina becomes regionally isolated and highly specialized. Thus, by placing adult hippocampal stem cell and progenitors (i.e., isolated from the adult hippocampus) into the developing and adult eye, these cells are anticipated to be surprisingly well suited for gene delivery.

Thus, in a particular aspect of the present invention there are provided herein methods for the transplantation of invention stem cells into diseased neural tissue. In application, the invention encompasses a method of treating dystrophic neural tissue, comprising introducing invention stem cells derived from an postmortem human donor into dystrophic neural tissue in a human recipient, e.g., by grafting or applying adult stem cells into tissue affected by the disorder. The recipient may be a young (immature) or an adult (mature).

Examples of dystrophic neural tissue that can be treated include the CNS tissue and neural tissue of the eye, particularly the retina or optic nerve. Thus, in another embodiment, the invention encompasses a method of repopulating or rescuing a dystrophic retina with neural cells, comprising introducing neural stem cells derived from an adult donor into dystrophic neural tissue of an animal recipient. The method is particularly useful for treating dystrophic retinal tissue caused by an optic neuropathy, e.g., glaucoma.

As used herein, the term “dystrophic neural tissue” encompasses damaged, injured, or diseased neural tissue, which neutral tissue includes differentiated neural tissue. Thus, provided herein are methods for treating a neuronal or neural disorder or neural injury. A “neuronal disorder” or “neural disorder” is any disorder or disease that involves the nervous system. One type of neuronal disorder is a neurodegenerative disorder. Neurodegenerative disorders include but are not limited to: (1) diseases of central motor systems including degenerative conditions affecting the basal ganglia (e.g., Huntington's disease, Wilson's disease, Striatonigral degeneration, corticobasal ganglionic degeneration, Tourettes syndrome, Parkinson's disease, progressive supranuclear palsy, progressive bulbar palsy, familial spastic paraplegia, spinomuscular atrophy, amyotrophic lateral sclerosis (ALS) and variants thereof, dentatorubral atrophy, olivo-pontocerebellar atrophy, paraneoplastic cerebellar degeneration, cerebral angiopathy (both hereditary and sporadic)); (2) diseases affecting sensory neurons (e.g., Friedreich's ataxia, diabetes, peripheral neuropathy, retinal neuronal degeneration); (3) diseases of limbic and cortical systems (e.g., s cerebral amyloidosis, Pick's atrophy, Retts syndrome; (4) neurodegenerative pathologies involving multiple neuronal systems and/or brainstem (e.g., Alzheimer's disease, autoimmune deficiency syndrome (AIDS) related dementia, Leigh's disease, diffuse Lewy body disease, epilepsy, Multiple system atrophy, Guillain-Barre syndrome, lysosomal storage disorders such as lipofuscinosis, late-degenerative stages of Down's syndrome, Alper's disease, vertigo as result of CNS degeneration; (5) pathologies arising with aging and chronic alcohol or drug abuse (e.g., with alcoholism the degeneration of neurons in locus oeruleus, cerebellum, cholinergic basal forebrain; with aging degeneration of cerebellar neurons and conical neurons leading to cognitive and motor impairments; and with chronic amphetamine abuse degeneration of basal ganglia neurons leading to motor impairments; and (6) pathological changes resulting from focal trauma such as stroke, focal ischemia, vascular insufficiency, hypoxic-ischemic encephalopathy, hyperglycemia, hypoglycemia or direct trauma.

The presence of a neuronal or neurodegenerative disorder or injury may be indicated by subjective symptoms, such as pain, change in sensation including decreased sensation, muscle weakness, coordination problems, imbalance, neurasthenia, malaise, decreased reaction times, tremors, confusion, poor memory, uncontrollable movement, lack of affect, obsessive/compulsive behavior, aphasia, agnosia, visual neglect, etc. Frequently, objective indicia, or signs observable by a physician or a health care provider, overlap with subjective indicia. Examples of objective indicia include the physician's observation of signs such as decreased reaction time, muscle fasciculations, tremors, rigidity, spasticity, muscle weakness, poor coordination, disorientation, dysphasia, dysarthria, and imbalance. Additionally, objective signs can include laboratory parameters, such as the assessment of neural tissue loss and function by positron emission tomography (PET) or functional magnetic resonance imaging (MRI), blood tests, biopsies and electrical studies such as electromyographic data.

The term “Treating” dystrophic neural tissue is intended to encompass repairing, replacing, augmenting, rescuing, or repopulating the diseased or damaged neural tissue, or otherwise compensating for the dystrophic condition of the neural tissue.

The term “Introduction” of invention stem cells into dystrophic neural tissue (e.g., a damaged or diseased nerve), may be accomplished by any means known in the medical arts, including but not limited to grafting and injection. It should be understood that such means of introducing the neural stem cells also encompass placing, injecting or grafting them into a site separate and/or apart from the diseased or damaged neural tissue site, since the neural stem cells are capable of migrating to and integrating into that dystrophic site. For example, dystrophic retinal or optic nerve tissue can be treated by placing neural stem cells into the vitreous of the eye.

Accordingly, provided herein are therapeutic methods comprising administering to a patient in need thereof an amount of the stem cells effective to repair or replace defective, damaged or dead tissue. In a presently preferred embodiment, cells which are to be added to or replaced comprise optic cells, including, retinal cells, Muller cells, amacrine cells, bipolar cells, horizontal cells, photoreceptors, astroglial cells, and the like.

Because of the pluripotent nature of the invention stem cells, and the resulting multiplicity of loci where such cells may be introduced in order to achieve therapeutic effects, there is a broad range of tissue damage and disease states that can be treated using the invention stem cells. Many disease states (e.g., liver disease) result in damaged or necrotic tissue. These types of diseases are ideal for replacement or augmentation therapy comprising the administration of the invention stem cells. The plastic and pluripotent nature of invention stem cells make them ideal candidates for their use as a source of cells which can be used to replace or correct for cells lost in disease or injury, even in the absence of exogenous genetic material.

For example, the invention stem cells can be used to replace a variety of tissue types throughout the body that are encompassed within the different phenotypes that progeny of invention stem cells can exhibit, upon differentiation, including glial cells, neurons, and the like. Accordingly, provided herein are therapeutic methods comprising administering to a patient in need thereof a cell population comprising invention stem cells as described herein, in an amount sufficient to provide a therapeutic effect.

The therapeutic benefit of these methods can be evaluated or assessed by any of a number of subjective or objective factors indicating a response of the condition being treated. Such indices include measures of increased neural or neuronal proliferation or more normal function of surviving brain areas. In addition, macroscopic methods of evaluating the effects of embodiments of the invention can be used which may be invasive or noninvasive. Further examples of evidence of a therapeutic benefit include clinical evaluations of cognitive functions including object identification, increased performance speed of defined tasks as compared to pretreatment performance speeds, and nerve conduction velocity studies.

Some disease states are characterized by one or more defective or missing genes. Such diseases are ideally treated by the administration of the invention stem cells containing one or more transgenes. Thus, provided herein are therapeutic methods wherein one or more disease-associated transgenes are incorporated and expressed in the invention stem cells following isolation of the postmortem cells and in vitro expansion. Examples of neuronal tissue-associated disease states and their associated genes include Huntingtons Corea (one or more of gamma amino butyric acid (GABA) decarboxyalse and CNTF), Alzheimer's disease (one or more of acetylcholinesterase, NGF, BDNF and FGF), Parkinson's disease (one or more of tyrosine hydroxylase, Dopa decarboxylase, GDNF, BDNF and FGF), ALS, and the like.

In another embodiment, the adipose-derived cells can be genetically modified, e.g., to express exogenous genes or to repress the expression of endogenous genes. In accordance with this embodiment, the cell is exposed to a gene transfer vector comprising a nucleic acid including a transgene, such that the nucleic acid is introduced into the cell under conditions appropriate for the transgene to be expressed within the cell. The transgene generally is an expression cassette, including a coding polynucleotide operably linked to a suitable promoter. The coding polynucleotide can encode a protein, or it can encode biologically active RNA, such as antisense RNA or a ribozyme. Thus, the coding polynucleotide can encode a gene conferring, for example, resistance to a toxin, a hormone (such as peptide growth hormones, hormone releasing factor, sex hormones, adrenocorticotrophic hormones, cytokines such as interferons, interleukins, and lymphokines), a cell surface-bound intracellular signaling moiety such as cell-adhesion molecules and hormone receptors, and factors promoting a given lineage of differentiation, or any other transgene with known sequence.

The expression cassette containing the transgene should be incorporated into the genetic vector suitable for delivering the transgene to the cell. Depending on the desired end application, any such vector can be so employed to genetically modify the cells (e.g., plasmids, naked DNA, viruses such as adenovirus, adeno-associated virus, herpesvirus, lentivirus, papillomavirus, retroviruses, etc.). Any method of constructing the desired expression cassette within such vectors can be employed, many of which are well known in the art, such as by direct cloning, homologous recombination, etc. The desired vector will largely determine the method used to introduce the vector into the cells, which are generally known in the art. Suitable techniques include protoplast fusion, calcium-phosphate precipitation, gene gun, electroporation, and infection with viral vectors.

The cells described herein can be used in combination with any known technique of tissue engineering, including but not limited to those technologies described in patents and publications cited in the Background of the Invention (including U.S. Pat. Nos. 5,902,741 and 5,863,531 to Advanced Tissue Sciences, Inc.) as well as, but not limited to: U.S. Pat. No. 6,139,574, Vacanti et al. (Oct. 31, 2000) Vascularized Tissue Regeneration Matrices Formed By Solid Free Form Fabrication Techniques; U.S. Pat. No. 5,759,830, Vacanti et al. (Jun. 2, 1998) Three-Dimensional Fibrous Scaffold Containing Attached Cells For Producing Vascularized Tissue In Vivo; U.S. Pat. No. 5,741,685, Vacanti, (Apr. 21. 1998) Parenchymal Cells Packaged In Immunoprotective Tissue For Implantation; U.S. Pat. No. 5,736,372, Vacanti et al. (Apr. 7, 1998) Biodegradable Synthetic Polymeric Fibrous Matrix Containing Chondrocyte For In Vivo Production Of A Cartilaginous Structure; U.S. Pat. No. 5,804,178, Vacanti et al. (Sep. 8, 1998) Implantation Of Cell-Matrix Structure Adjacent Mesentery, Omentum Or Peritoneum Tissue; U.S. Pat. No. 5,770,417, Vacanti et al. (Jun. 23. 1998) Three-Dimensional Fibrous Scaffold Containing Attached Cells For Producing Vascularized Tissue In Vivo; U.S. Pat. No. 5,770,193, Vacanti et al. (Jun. 23. 1998) Preparation of Three-Dimensional Fibrous Scaffold For Attaching Cells To Produce Vascularized Tissue In Vivo; U.S. Pat. No. 5,709,854, Griffith-Cima et al. (Jan. 20, 1998) Tissue Formation By Injecting A Cell-Polymeric Solution That Gels In Vivo; U.S. Pat. No. 5,516,532, Atala et al. (May 14, 1998) Injectable Non-Immunogenic Cartilage And Bone Preparation; U.S. Pat. No. 5,855,610, Vacanti et al. (Jan. 5. 1999) Engineering Of Strong, Pliable Tissues; U.S. Pat. No. 5,041,138, Vacanti et al. (Aug. 20, 1991) Neomorphogenesis Of Cartilage In Vivo From Cell Culture; U.S. Pat. No. 6,027,744, Vacanti et al. (Feb. 22, 1900) Guided Development and Support Of Hydrogel-Cell Compositions; U.S. Pat. No. 6,123,727, Vacanti et al. (Sep. 26, 2000) Tissue Engineered Tendons And Ligament; U.S. Pat. No. 5,536,656, Kemp et al. (Jul. 16, 1996) Preparation Of Tissue Equivalents By Contraction Of A Collagen Gel Layered On A Collagen Gel; U.S. Pat. No. 5,144,016, Skjak-Braek et al. (Sep. 1, 1992) Alginate Gels; U.S. Pat. No. 5,944,754, Vacanti (Aug. 31, 1999) Tissue Re-Surfacing With Hydrogel-Cell Compositions; U.S. Pat. No. 5,723,331, Tubo et al. (Mar. 3, 1998) Methods And Compositions For The Repair Of Articular Cartilage Defects In Mammals; U.S. Pat. No. 6,143,501, Sittinger et al. (Nov. 7, 2000) Artificial Tissues, Methods For The Production And The Use Thereof.

While the invention stem cells are useful to introduce therapeutic genes, it may be desirable to introduce into a host or patient one or more genes that are not strictly therapeutic but which may be useful in other ways, for example, as tracking genes (i.e., markers), as genes to induce migration, as genes to induce mitosis, as survival genes, as suicide genes, and the like. Marker genes contemplated for use in conjunction with the invention stem cells include genes encoding a modified green fluorescent protein (GFP) derived from jellyfish, β-Galatosidase (the LacZ gene product), neomycin phosphotransferase (neo), Luciferase, and the like.

As will be recognized by those of skill in the art, a variety of methods exist for the introduction of genetic material into cells such as the invention stem cells. Such methods include viral and non-viral methods. Non-viral methods contemplated for introducing genetic material into cells include electroporation, microinjection, polyethylene glycol precipitation, high velocity ballistic penetration by small particles with the nucleic acid to be introduced contained either within the matrix of such particles, or on the surface thereof (Klein, et al (1987), Nature 327, 70), or the like. Viral methods contemplated for introducing genetic material into cells include the use of retroviral vectors, and the like. It is presently preferred that retroviral vectors be employed for introducing genetic material into the invention stem cells. In particular, replication deficient vectors can be employed. Such vectors are well known to those of skill in the art.

Numerous examples of non-neural diseases exist that are also suitable for treatment with the invention stem cells. Some of these disease states are equally suited for treatment using the invention stem cells with and/or without incorporated transgenes. For example, the liver plays a central role in the pathophysiology of many inherited metabolic diseases. Although the adult liver has the unusual ability to regenerate after injury, the liver is an important target for cell therapy. For example, invention stem cells can be introduced into the liver where they differentiate into hepatocytes, and replace dead and dying cells, thereby correcting disease phenotypes. When particular diseases are associated with one or more missing or defective genes, such diseases are treatable with the invention stem cells wherein the missing/defective gene(s) is/are incorporated.

Recent experimental data from immune and endocrine studies using spontaneous or transgenic models of diabetes have emphasized the role of islets of Langerhans, and particularly beta cells, in autoimmune insulin-dependent (Type 1) diabetes mellitus (IDDM) pathogenesis. IDDM is a chronic disorder that results from the destruction of the insulin-producing beta cells of the pancreatic islets. Accordingly, invention stem cells are grafted in the pancreas for the replacement of damaged pancreas cells with the grafted cells. When particular diabetic pathologies are associated with one or more missing or defective genes, such pathologies are treatable with the invention stem cells wherein the missing/defective gene(s) is/are incorporated.

Duchenne muscular dystrophy (DMD) is characterized by slow and progressive muscle weakness affecting limb and respiratory muscles, which degenerate until fatal cardiorespiratory failure. Myodystrophy of the Duchenne type results from mutations affecting the gene for dystrophin, a cytoskeletal protein. A form of congenital dystrophy caused by a deficiency of the a2 subunit of the basement membrane protein laminin/merosin is termed Merosin-Deficient Congenital Muscular Dystrophy (MCMD). Accordingly, invention stem cells are grafted into muscles wherein they differentiate to become myoblasts and replace degenerating muscle cells.

Cardiac disease, typified in many instances by damaged heart muscle, is another target for cell replacement. Accordingly, invention stem cells are transplanted into the heart to replace diseased cells and improve heart function.

Pulmonary disease (i.e., Cystic fibrosis) is the most common autosomally inherited disease, and is caused by the defective gene Cftr, which encodes an ion channel at the cell membrane. Augmentation of lung tissue with invention stem cells can alleviate the reduced respiratory function caused by the defective genotype. Accordingly, invention stem cells are grafted into the lung in order to replace the diseased cells having defective ion channels, and restore normal lung function. As a heritable disorder, this disease is also an ideal candidate for treatment using the invention stem cells with appropriately incorporated Cftr-augmenting exogenous nucleic acids.

As readily understood by those of skill in the art, the most direct method for administration of the invention stem cells to the desired site is likely to be by injection. However, any means of administering cells that results in correct localization and integration is contemplated for use in patients in need of the invention stem cells.

As those of skill in the art will understand, a number of factors may be determinative of when and how a stem or progenitor cell differentiates. As a result, it may be desirable to induce differentiation of the invention stem cells in a controlled manner and/or by employing factors which are not easily or desirably introduced into the locus of therapeutic invention stem cells introduction. Accordingly, there are provided therapeutic methods as described herein, wherein said invention stem cells have been induced to differentiate, prior to administration to the subject, by in vitro exposure to extracellular and/or intracellular factors described herein, including trophic factors, cytokines, mitogens, hormones, cognate receptors for the foregoing, and the like, as well as combinations of any two or more thereof.

### Kits

The invention provides for a postmortem tissue culture kit comprising, in suitable container, base culture medium reagents in suitable quantities to formulate a base culture medium and at least one trophic factor in a suitable quantity to formulate a stem cell differentiating concentration of said at least one trophic factors and/or a cofactor therefore, e.g., a suitable quantity of glycosylated cystatin C. Preferably, the kit will contain a cocktail of trophic factors, including at least one neurotrophin.

The kit may comprise a single container means that contains the base medium reagents, the at least one trophic factor, and optionally, a cofactor therefore, e.g., a suitable quantity of glycosylated cystatin C. The container means may, if desired, contain a pharmaceutically acceptable sterile solvent, such as water or saline, having associated with it, the base medium reagents, the at least one trophic factor, and optionally, a cofactor therefore, e.g., a suitable quantity of glycosylated cystatin C. The formulation may be in the form of a gelatinous composition (e.g. a collagenous composition), a powder, solution, matrix, lyophilized reagent, or any other such suitable means. The single container means may contain a dry, or lyophilized mixture of reagents, trophic factors, and other components.

Alternatively, the kits of the invention may comprise distinct container means for each component. In such cases, one or more containers would contain each of the base culture medium reagents, the at least one trophic factors, and optionally the glycosylated cystatin C, either as sterile solutions, powders, lyophilized forms, etc.

The kits may also comprise a second or third container means for containing a sterile, pharmaceutically acceptable buffer, diluent or solvent. Such a solution may, be required to formulate the postmortem tissue culture medium kit components into a more suitable form for application. It should be noted, however, that all components of a kit could be supplied in a dry form (lyophilized), which would allow for “wetting” upon contact with a suitable fluid. Thus, the presence of any type of pharmaceutically acceptable buffer or solvent is not a requirement for the kits of the invention.

The container means will generally be a container such as a vial, test tube, flask, bottle, syringe or other container means, into which the components of the kit may placed. The medium components may also be aliquoted into smaller containers, should this be desired. The kits of the present invention may also include a means for containing the individual containers in close confinement for commercial sale, such as, e.g., injection or blow-molded plastic containers into which the desired vials or syringes are retained.

### EXAMPLES

The following examples are put forth so as to provide those of ordinary skill in the art with a complete disclosure and description of how to make and use the subject invention, and are not intended to limit the scope of what is regarded as the invention. Efforts have been made to ensure accuracy with respect to the numbers used (e.g. amounts, temperature, concentrations, etc.) but some experimental errors and deviations should be allowed for. Unless otherwise indicated, parts are parts by weight, molecular weight is average molecular weight, temperature is in degrees centigrade; and pressure is at or near atmospheric.

All publications and patent applications cited in this specification are herein incorporated by reference as if each individual publication or patent application were specifically and individually indicated to be incorporated by reference. The citation of any publication is for its disclosure prior to the filing date and should not be construed as an16admission that the present invention is not entitled to antedate such publication by virtue of prior invention.

It is to be understood that this invention is not limited to the particular methodology, protocols, cell lines, animal species or genera, and reagents described, as such may vary.

It is also to be understood that the terminology used herein is for the purpose of describing particular embodiments only, and is not intended to limit the scope of the present invention which will be limited only by the appended claims.

As used herein the singular forms “a”, “and”, and “the” include plural referents unless the context clearly dictates otherwise. Thus, for example, reference to “a cell” includes a plurality of such cells and reference to “the protein” includes reference to one or more proteins and equivalents thereof known to those skilled in the art, and so forth. All technical and scientific terms used herein have the same meaning as commonly understood to one of ordinary skill in the art to which this invention belongs unless clearly indicated otherwise.

### Example 1

### Expansion and Propagation in Neonatal and Adult Tissue

Brain tissue from an 11-week-old postnatal male who died of extracerebral complications of myofibromatosis, and a 27 year-old male temporal cortex resection were used. The post-mortem tissue was removed and sectioned 2 hours after death, placed in cold, antibiotic-containing, Hank's buffered salt solution and then processed for culture 3 hours later. Representative sections of hippocampus, subventricular zone, motor cortex, and corpus callosum were taken. The temporal cortex tissue was provided enblock and placed into chilled Hank's buffered salt solution and processed for culture 3 hours after removal. The adult tissues were divided into hippocampal formation, white matter, and remaining cortical gray matter. Tissues were finely diced, dissociated with papain, DNase I, and neutral protease for 45 minutes at 37° C. as previously described for rodent tissues (Palmer, T. D., Markakis, E. A., Willhoite, A. R., Safar, F. & Gage, F. H. (1999) J. Neurosci. 19, 8487-8497).

Isolated cells were initially plated onto fibronectin coated plates in Dulbecco/Vogt modified Eagle's minimal essential medium (DMEM):F-12 (1:1) medium containing glutamine, amphotericin-B, penicillin, streptomycin, and 10% fetal bovine serum. After 24 hours, the medium was replaced with DMEM:F12 supplemented with BIT-9500 (bovine serum albumin, transferin, insulin; Stem Cell Technologies, Vancouver BC), 20 ng/ml basic fibroblast growth factor (FGF-2), 20 ng/ml epidermal growth factor, 20 ng/ml platelet-derived growth factor-ab. This medium was further supplemented by 25% conditioned medium from rat stem cells genetically modified to overproduce a secreted form of FGF-2 and its stem cell co-factor the glycosylated form of Cystatin C (CCg) (Taupin P. et al. (2000) Neuron 28, 385-397) The conditioned medium significantly improved overall growth and plating efficiency. The medium was changed every two days and cultures replated onto twice the surface area as needed to accommodate proliferative expansion. All tissue samples yielded neural progenitor cells but the highest yields were from hippocampus and ventricular zone. For long-term storage, cultures were dissociated with trypsin, rinsed, and cryopreserved in growth medium (without growth factors) containing 10% dimethyl sulfoxide (DMSO).

Cells from the 11-week old tissue grew at log phase for more than 70 population doublings before showing signs of in vitro senescence (significant reduction in growth rates). The adult tissues were expanded for more than 30 doublings before senescence. Neurons were spontaneously generated at all stages in the cultures and more complete differentiation could be induced by growth factor withdrawal and stimulation with forskolin and retinoic acid. Neonatal and adult cultures produced similar numbers of neurons and astrocytes.

At present, 23 human tissue samples have been processed from diverse age groups. Most samples have yielded viable progenitor cells, with the longest post mortem interval being about 20 hours. Tissues from young individuals have yielded significantly more cells per gram and these cells display a higher proliferative capacity.

### Example 2

### Fetal, Neonatal, and Adult Tissue Preparation

Informed consent for the donation of fetal, pediatric and adult brain tissue was acquired prior to tissue acquisition. All tissues were acquired in compliance with National Institutes of Health (NIH) and institutional guidelines. For the present study the brain tissue from a 16 wk abortis, 11-week-old postnatal male who died of extracerebral complications of myofibromatosis, and an adult temporal cortex resection were used. The post-mortem tissue was removed in a standard autopsy manner that had been modified to allow aseptic conditions. Tissues were sectioned, rinsed twice and then placed in sterile, ice-cold, antibiotic-containing, Hank's buffered salt solution. Whole brain tissue was used from fetal brain. Representative sections of hippocampus, subventricular zone, motorcortex, and corpus callosum were taken from the neonatal brain. The temporal cortex resection was divided into hippocampal formation and white matter.

Tissues were diced with scalpel blades and then incubated with mixing in an enzyme solution containing papain, DNase I, and neutral protease. Partially digested tissues were further dissociated by centrifugation and resuspension in serum-containing medium. Whole tissue dissociates were either plated directly or further fractionated by separation over a 45% Percoll gradient. Cells were initially plated onto fibronectin coated platesin DMEM:Ham's F-12 medium containing glutamine, gentamicin, amphotericin, penicillin, streptomycin, and fetal bovine serum. After 24 hours, the medium was replaced with DMEM:F12 containing 1 mg/ml bovine serum albumin, 5 ug/ml insulin, 20 ng/ml basic fibroblast growth factor (FGF-2), 20 ng/ml epidermal growth factor (EGF), and 20 ng/ml platelet-derived growth factor-ab (PDGFab). This basal growth medium was further supplemented by 25% conditioned medium from rat stem cells genetically modified to overproduce FGF-2 and its stem cell co-factor the glycosylated form of Cystatin C (CCg) (Taupin P. et al. (2000) Neuron 28, 385-397). The conditioned medium significantly improved overall growth and plating efficiency. The medium was changed every two days and, at confluence, cultures were passaged as needed until about 150 square cm of confluent culture were produced. These cultures were lifted with trypsin, rinsed, and cryopreserved in medium containing 1 mg/ml BSA, insulin, transferin and 10% DMSO.

### Example 3

### Staining, Counting, and Plating

Progenitor cells were isolated from postmortem tissues of an 11-week-old male and cultured for approximately 20 population doublings. Proliferative progenitor cells were antibody stained for type III-β tubulin (green), glial fibrillary acidic protein (GFAP, red), and DAPI nuclei stain (blue).

In order to stain with antibodies, the cells were fixed with 4% phosphate buffered paraformaldehyde for 10 minutes at room temperature and then rinsed twice with phosphate buffered saline (PBS). Samples were then blocked for 30′ in PBS, 0.3% triton X-100, 10% pre-immune serum of the same species and isotype as the secondary antibody used (PBS++). The samples were then incubated overnight with primary antibodies diluted in PBS++. Samples were then rinsed 3 times with PBS and incubated overnight with fluorescent secondary antibodies in PBS++. Samples were then washed and cover-slipped in 10% glycerol, 10% polyvinyl alcohol in PBS.

All three colors (green, red, and blue) were displayed indicating the presence of type III-β tubulin, GFAP, and nuclei. Differentiated cells were stained for Map2 (red), neurofilament 150 (green) resulting yellow for double labeled neurons, GFAP (blue), and nuclei (white). Using the staining methods described, differentiated cells were observed and highlighted in red, green, yellow, blue, and white. Cells were plated at low density onto polyornithine/laminin coated dishes and treated for 7 days with DMEM:F12 containing 1% FBS, 100 nM all-trans retinoic acid, 5 uM forskolin and 2 ng/ml FGF-2.

Phase contrast images were taken after 7, 14, and 21 days in culture on a primary culture from a temporal cortex biopsy taken from a 27 year-old male. At each passage, neural progenitor cells taken from neonatal (11 week) and adult (27 years) tissue were counted and plated at approximately the original density onto new dishes. Cell number and population doublings were corrected for plating efficiency.

Cells taken from neonatal (at passage 12) or adult (at passage 8) tissue were allowed to differentiate and then immunostained and scored for the indicated markers (phenotype). Type III β tubulin (green) marked immature neurons, NeuN marked postmitotic neurons, GFAP (red) marked astrocytes, 04 marked immature oligodendrocytes and fibronectin marked fibroblasts and connective tissue.

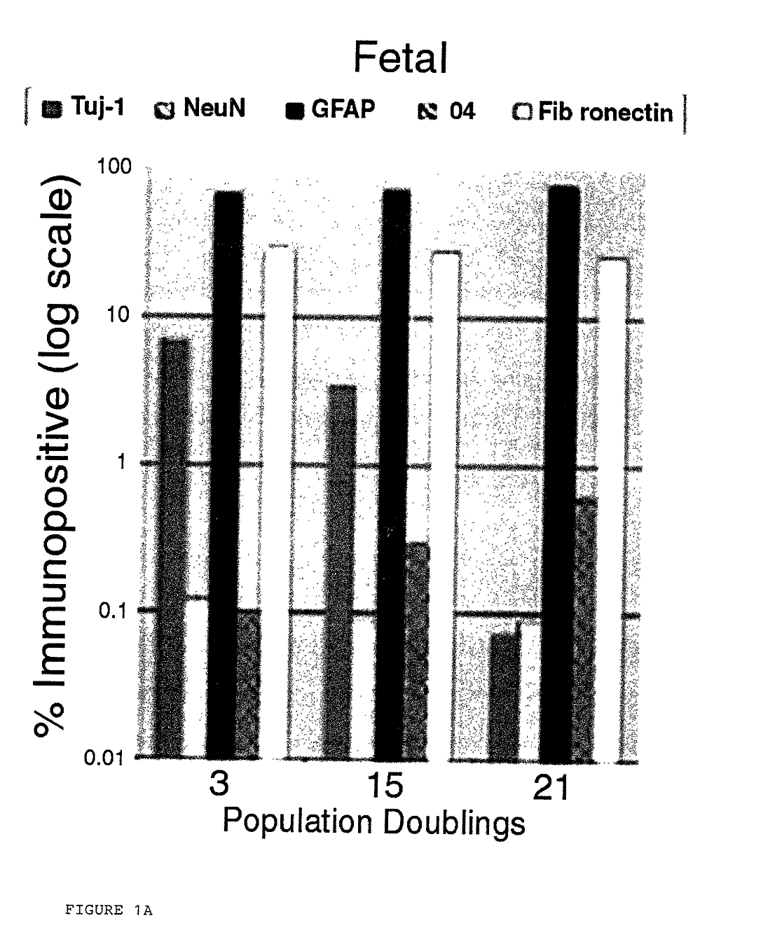

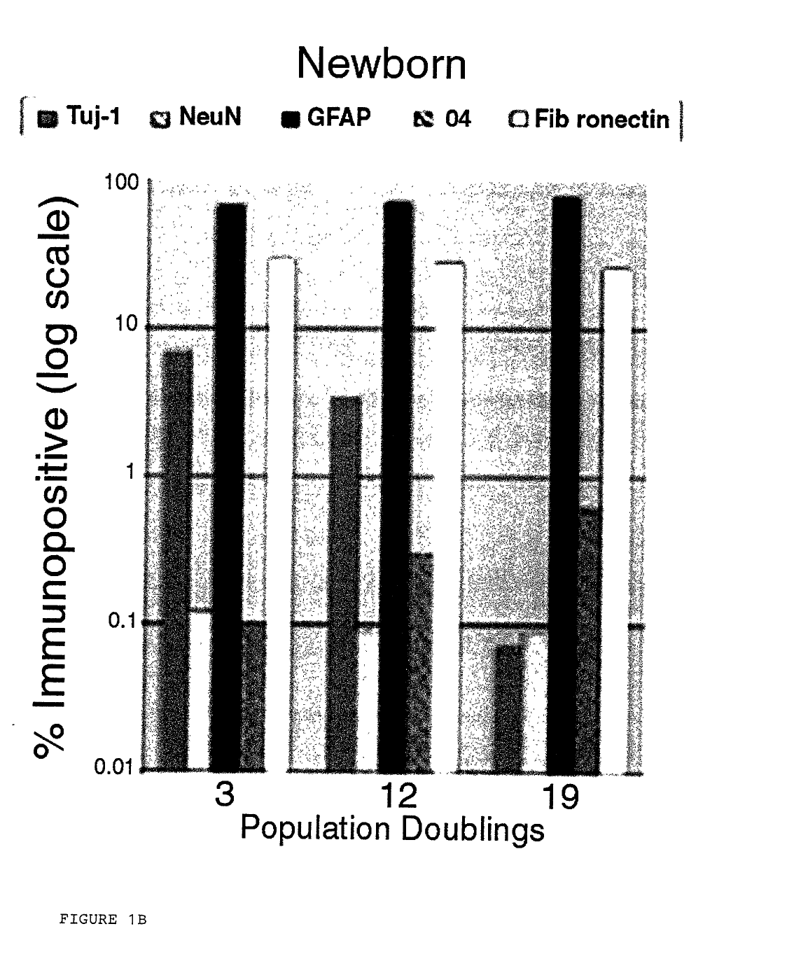

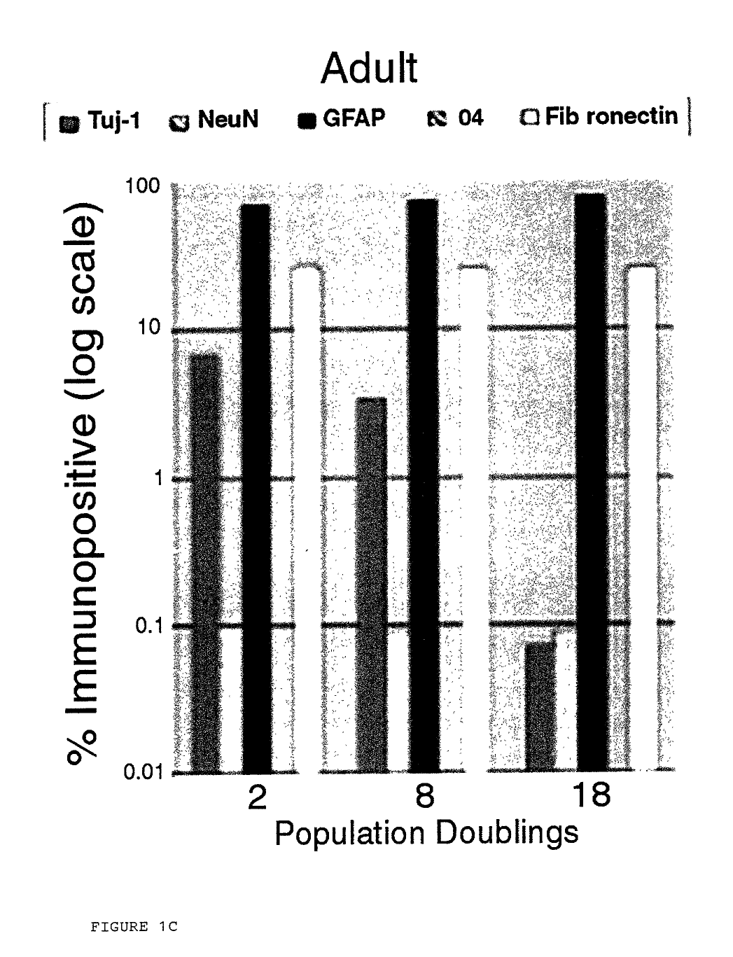

Differentiated cells were found to express markers indicating Neurons (Tuj-1, NeuN), Astrocytes (GFAP), and Oligodendrocytes (O4). All three cell types were detectable in cultures from fetal (FIG. 1A), newborn (FIG. 1B) or adult (FIG. 1C) brain tissues. The number of neurons generated decreased over time while the number of glia remained relatively constant. Fetal cultures were derived from whole brain less cerebellum and brain stem. Hippocampal tissue was used to initiate the newborn and adult cultures scored here.

### Example 5

### Growth Rates

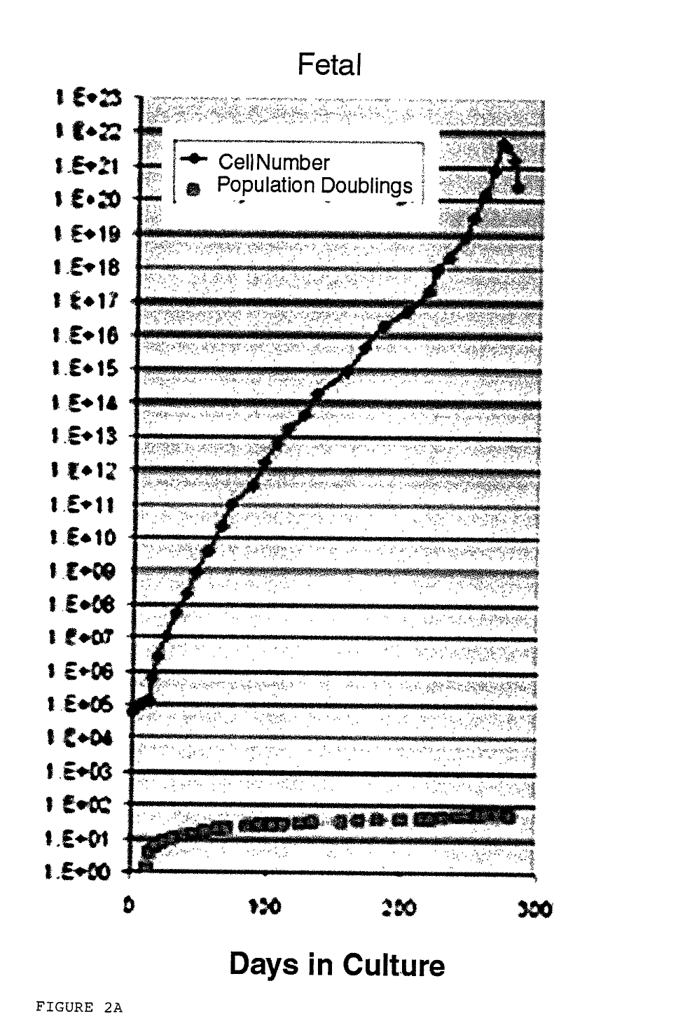

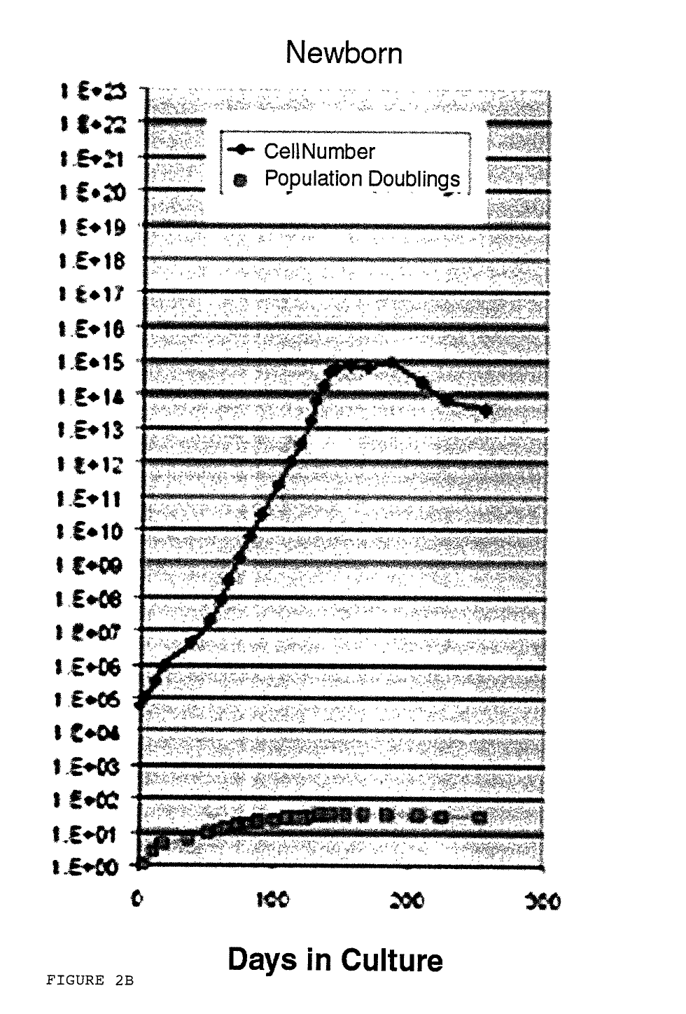

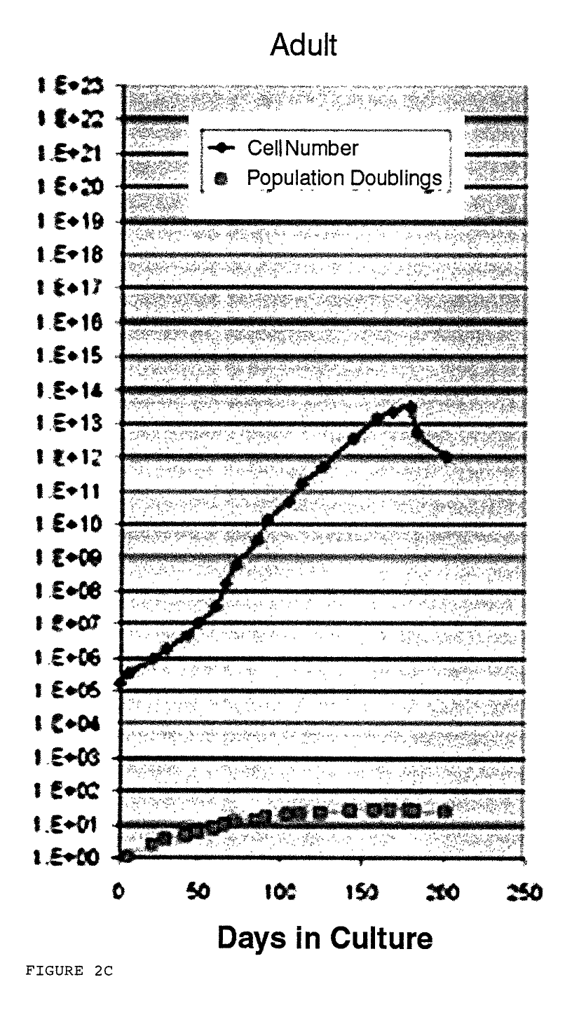

Representative primary cultures from fetal (FIG. 2A), newborn (FIG. 2B) or adult (FIG. 2C) brain tissues showed stable growth rates up to the point of senescence. Fetal cells show optimum growth for approximately 40 population doublings. Cells from neonatal tissues rapidly divide for approximately 30 doublings and adult tissues for approximately 25 doublings. Cell number was extrapolated by counting cells at each passage and adjusting the calculated growth for plating efficiency.

While the present invention has been described in some detail for purposes of clarity and understanding, one skilled in the art will appreciate that various changes in form and detail can be made without departing from the true scope of the invention. All references referred to above are hereby incorporated by reference.

What is claimed is:

1. A tissue culture medium for propagating postmortem stem cells comprising:
a base culture medium and a stem cell differentiating concentration of at least one trophic factors.

2. The tissue culture medium of claim 1, wherein the base culture medium is a defined medium.

3. The tissue culture medium of claim 2, wherein the defined medium is selected from the group consisting of Minimum Essential Medium Eagle, ADC-1, LPM (Bovine Serum Albumin-free), F10(HAM), F12 (HAM), DCCM1, DCCM2, RPMI 1640, BGJ Medium (with and without Fitton-Jackson Modification), Basal Medium Eagle (BME-with the addition of Earle's salt base), Dulbecco's Modified Eagle Medium (DMEM-without serum), Yamane, IMEM-20, Glasgow Modification Eagle Medium (GMEM), Leibovitz L-15 Medium, McCoy's 5A Medium, Medium M199 (M199E-with Earle's sale base), Medium M199 (M199H-with Hank's salt base), Minimum Essential Medium Eagle (MEM-E-with Earle's salt base), Minimum Essential Medium Eagle (MEM-H-with Hank's salt base) and Minimum Essential Medium Eagle (MEM-NAA with non essential amino acids), medium 199, CMRL 1415, CMRL 1969, CMRL 1066, NCTC 135, MB 75261, MAB 8713, DM 145, Williams' G, Neuman & Tytell, Higuchi, MCDB 301, MCDB 202, MCDB 501, MCDB 401, MCDB 411, and MDBC 153.

4. The tissue culture medium of claim 2, wherein the defined medium is Dulbecc/Vogt modified Eagle's minimal essential medium (DMEM):F12 medium at a 1:1 ratio.

5. The tissue culture medium of claim 1, wherein said trophic factor is a growth factor.

6. The tissue culture medium of claim 5, wherein said growth factor is selected from the group consisting of epidermal growth factor (EGF), platelet derived growth factor (PDGF), and Fibroblast Growth Factor-2 (FGF-2).

7. The tissue culture medium of claim 1, wherein the trophic factor comprises a trophic co-factor.

8. The tissue culture medium of claim 8, further comprising glycosylated cystatin C.

9. The tissue culture medium of claim 1, wherein said trophic factor comprises a combination of Fibroblast Growth Factor-2 (FGF-2), platelet derived growth factor (PDGF), epidermal growth factor (EGF), and glycosylated cystatin C.

10. A postmortem tissue culture medium kit comprising in a suitable container:
base culture medium reagents in suitable quantities to formulate a base culture medium; and
at least one trophic factors in a suitable quantity to formulate a stem cell differentiating concentration of said at least one trophic factors.

11. The postmortem tissue culture medium kit of claim 10, wherein the base culture medium is a defined medium.

12. The postmortem tissue culture medium kit of claim 11, wherein the defined medium is selected from the group consisting of Minimum Essential Medium Eagle, ADC-1, LPM (Bovine Serum Albumin-free), F10(HAM), F12 (HAM), DCCM1, DCCM2, RPMI 1640, BGJ. Medium (with and without Fitton-Jackson Modification), Basal Medium Eagle (BME-with the addition of Earle's salt base), Dulbecco's Modified Eagle Medium (DMEM-without serum), Yamane, IMEM-20, Glasgow Modification Eagle Medium (GMEM), Leibovitz L-15 Medium, McCoy's 5A Medium, Medium M199 (M199E-with Earle's sale base), Medium M199 (M199H-with Hank's salt base), Minimum Essential Medium Eagle (MEM-E-with Earle's salt base), Minimum Essential Medium Eagle (MEM-H-with Hank's salt base) and Minimum Essential Medium Eagle (MEM-NAA with non essential amino acids), medium 199, CMRL 1415, CMRL 1969, CMRL 1066, NCTC 135, MB 75261, MAB 8713, DM 145, Williams' G, Neuman & Tytell, Higuchi, MCDB 301, MCDB 202, MCDB 501, MCDB 401, MCDB 411, and MDBC 153.

13. The postmortem tissue culture medium kit of claim 11, wherein the defined medium is Dulbecco/Vogt modified Eagle's minimal essential medium (DMEM):F12 medium at a 1:1 ratio.

14. The postmortem tissue culture medium kit of claim 10, wherein said trophic factor is a growth factor.

15. The postmortem tissue culture medium kit of claim 14, wherein said growth factor is is selected from the group consisting of epidermal growth factor (EGF), platelet derived growth factor (PDGF), and Fibroblast Growth Factor-2 (FGF-2).

16. The postmortem tissue culture medium kit of claim 10, further comprising glycosylated cystatin C.

17. The postmortem tissue culture medium kit of claim 16, wherein said trophic factor comprises a combination of Fibroblast Growth Factor-2 (FGF-2), platelet derived growth factor (PDGF) and epidermal growth factor (EGF).

18. A postmortem stem cell, wherein the postmortem stem cell is derived by growing a postmortem cell sample in the culture medium of claim 1.

19. The postmortem stem cell of claim 18, wherein said postmortem cell sample comprises brain cells.

20. The postmortem stem cell of claim 19, wherein the brain cells are derived from the temporal cortex, hippocampus or the ventricular zone of the brain.

21. The postmortem stem cell of claim 18, wherein said postmortem cell sample comprise neural precursor cells.

22. The postmortem stem cell of claim 18, wherein said postmortem cell sample is cultured in the presence of conditioned media taken from stem cells that produce recombinant glycosylated cystatin C.

23. The postmortem stem cell of claim 18, wherein said postmortem cell sample is grown for greater than about 70 population doublings.

24. The postmortem stem cell of claim 18, wherein said postmortem cell sample is taken from the host more than about 20 hours following death.

25. A method of growing postmortem cells in culture, comprising:
providing postmortem cells; and
culturing said postmortem cells in the presence of a trophic factor and glycosylated cystatin C.

26. The method of claim 25, wherein said postmortem cells are brain cells.

27. The method of claim 27, wherein the brain cells are derived from the temporal cortex, hippocampus or the ventricular zone of the brain.

28. The method of claim 25, wherein said postmortem cells are neural precursor cells.

29. The method of claim 25, comprising culturing said postmortem cells in the presence of conditioned media taken from stem cells that produce recombinant glycosylated cystatin C.

30. The method of claim 25, wherein said trophic factor is selected from the group consisting of epidermal growth factor (EGF), platelet derived growth factor (PDGF), and Fibroblast Growth Factor-2 (FGF-2).

31. The method of claim 25, wherein said trophic factor comprises a combination of Fibroblast Growth Factor-2 (FGF-2), platelet derived growth factor (PDGF), epidermal growth factor (EGF), and glycosylated cystatin C.

32. The method of claim 25, wherein said postmortem cells are grown for greater than about 70 population doublings.

33. The method of claim 25, wherein the postmortem cells are taken more than about 20 hours following death.
